(19) United States
(12) Patent Application Publication
(10) Pub. No.: US 2023/0181168 A1
(43) Pub. Date: Jun. 15, 2023

(54) DISTRIBUTED ULTRASONIC SCANNING SYSTEM AND COMMUNICATION METHOD

(71) Applicant: Ruijin Hospital, Shanghai Jiaotong University School of Medicine, Shanghai (CN)

(72) Inventor: Jianqiao ZHOU, Shanghai (CN)

(21) Appl. No.: 17/695,573

(22) Filed: Mar. 15, 2022

(30) Foreign Application Priority Data
Dec. 15, 2021 (CN) 202111538537.X

(51) Int. Cl. A61B 8/00; G06T 7/00; A61B 8/08

(52) U.S. Cl. CPC A61B 8/565; G06T 7/0012; A61B 8/5207; A61B 8/461; A61B 8/085; A61B 8/5223; G06T 2207/10132; G06T 2207/30096

(57) ABSTRACT

The invention relates to the technical field of medical image processing, and more particularly, to a distributed ultrasonic scanning system and a communication method. The method comprises at least one ultrasonic scanning device, comprising at least one scanning probe for performing an ultrasonic scanning operation on a patient to obtain echo data; a data acquisition unit, connected to the at least one ultrasonic scanning device, configured to receive the echo data and to compress the echo data to generate compressed data; and a processing host, connected to the data acquisition unit, configured to obtain the compressed data and to generate a first ultrasonic image according to the compressed data. The present invention has the beneficial effects that a data acquisition unit is provided to enable ultrasonic image data to effectively flow in the network, so that the problem of a poor flow of the image is avoided.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### RELATED APPLICATION INFORMATION

This patent claims priority from Chinese Patent Application No. 202111538537.X, filed Dec. 15, 2021 entitled, “DISTRIBUTED ULTRASONIC SCANNING SYSTEM AND COMMUNICATION METHOD” the entirety of which is incorporated herein by reference.

### BACKGROUND

The invention relates to the technical field of medical image processing, and more particularly, to a distributed ultrasonic scanning system and a communication method.

### DESCRIPTION OF THE RELATED ART

Medical ultrasound testing is an ultrasound-based medical diagnostic imaging technique. It is used to visualize patient's muscles, internal organs including large or small body structures, and pathology lesions in vitro, assisting a doctor in making an effective diagnosis for the patient's disease. An ultrasonic scanning device is a medical instrument with which the above-mentioned technique is applied. The device detects all body structures of the patient by transmitting ultrasonic waves and detecting the echo of the waves, so as to assist the doctor with the medical diagnosis. Generally, the ultrasonic scanning device comprises at least one scanning probe, and at least one transducer is provided in the scanning probe for emitting ultrasonic waves to the patient, and echo of the ultrasonic waves are received by the at least one scanning probe. The ultrasonic scanning device can be generally divided into the following categories, for example, A-type ultrasonic detection, B-type ultrasonic detection, D-type ultrasonic detection, M-type ultrasonic detection, ultrasound elastography, and contrast-enhanced ultrasound imaging according to their detection methods. The B-type ultrasonic detection, a grayscale ultrasound image, is the most commonly used ultrasonic testing means in the medical examination.

In the prior art, since the image reconstruction of the ultrasonic scanning device needs to occupy a lot of computing resources, the existing ultrasonic scanning device usually comprises a large host to process the received ultrasonic echoes. Thus, the ultrasonic scanning device is usually arranged in a specific location and it is hard to move. At the same time, in practice, the inventor found that due to a poor compatibility design between ultrasonic scanning devices in different ages and from different manufacturers, the existing ultrasonic scanning data flow was not smooth, and it could not well meet the requirements of medical digitization. In addition, it is difficult to use artificial intelligence technology to further process images, which leads to the problem of poor diagnosis and treatment. Furthermore, since the existing ultrasonic scanning device can usually only be set up in a specific location, when a patient needs an ultrasound scan, he/she has to go to a healthcare institution, so it cannot well meet the needs for some patients to perform ultrasonic scanning operations at home or in the community.

### SUMMARY

Given that the foregoing problems exist in the prior art, the present invention provides a distributed ultrasonic scanning system and a communication method.

A detailed technical solution is as follows:
a distributed ultrasonic scanning system, comprising:
at least one ultrasonic scanning device, comprising at least one scanning probe for performing an ultrasonic scanning operation on a patient to obtain echo data;
a data acquisition unit, connected to the at least one ultrasonic scanning device, configured to receive the echo data and to compress the echo data to generate compressed data; and
a processing host, connected to the data acquisition unit, configured to obtain the compressed data and to generate a first ultrasonic image according to the compressed data.

Preferably, the ultrasonic scanning system further comprises a terminal processing device;
wherein the terminal processing device is connected to the data acquisition unit or the ultrasonic scanning device to obtain the echo data;
the terminal processing device generates and displays a second ultrasonic image according to the echo data; and
an imaging quality of the second ultrasonic image is lower than that of the first ultrasonic image.

Preferably, the terminal processing device comprises:
a communication module, connected to the ultrasonic scanning device or the data acquisition unit, and configured to receive the echo data;
a loop detection module, connected to the communication module;
when the communication module receives the echo data from the ultrasonic scanning device, the loop detection module detects a communication connection status between the communication module and the ultrasonic scanning device;
when the communication connection status reaches a preset condition, the loop detection module controls the communication module to establish a communication connection with the data acquisition unit, so as to continue to receive the echo data.

Preferably, the ultrasonic scanning system further comprises:
an information management unit, connected to the processing host, and configured to obtain the first ultrasonic image;
wherein the information management unit comprises a medical record sub-module, the medical record sub-module records the first ultrasonic image in a pre-collected electronic medical record corresponding to the patient.

Preferably, the ultrasonic scanning system further comprises an image recognition unit;
wherein the image recognition unit is connected to the information management unit for obtaining the first ultrasonic image from the information management unit;
the image recognition unit comprises:
a lesion identification sub-module, configured to determine the presence of a lesion in the first ultrasonic image based on the first ultrasonic image;
a lesion segmentation sub-module, connected to the lesion identification sub-module;
when the lesion is present in the first ultrasonic image, the lesion segmentation sub-module segments an image of the lesion from the first ultrasonic image and stores the segmented lesion image in the information management unit.

Preferably, the ultrasonic scanning system further comprises an auxiliary diagnostic unit;
wherein the auxiliary diagnostic unit is connected to the information management unit for obtaining the lesion image from the information management unit;
the auxiliary diagnostic unit comprises an identification sub-module for identifying the lesion in the lesion image to generate classification information;
the medical record sub-module in the information management unit receives and stores the classification information, and the classification information is used to assist the doctor in diagnosing the patient.

Preferably, the information management unit is connected to a plurality of medical terminals;
the information management unit selects a corresponding one of the plurality of medical terminals according to the electronic medical record and a pre-entered diagnosis process, so as to distribute at least one data of the electronic medical record, the first ultrasonic image and the lesion image.

Preferably, each of the plurality of medical terminals has a corresponding healthcare institution identifier and a hospital department identifier;
wherein the information management unit comprises a labeling sub-module, the labeling sub-module obtains the healthcare institution identifier and the hospital department identifier of one of the plurality of medical terminals, and selects, based on the healthcare institution identifier and the hospital department identifier, a corresponding process flow to process the first ultrasonic image; and
the information management unit distributes the processed first ultrasonic image to the medical terminals.

A communication method applicable for the ultrasonic scanning system as described above, wherein the data acquisition unit in the ultrasonic scanning system is connected to the processing host through a plurality of gateway devices, the communication method comprises:
Step S1, obtaining a network identity of the data acquisition unit when the data acquisition unit obtains the echo data;
Step S2, obtaining at least two processing hosts based on the network identity and a pre-generated network topology;
Step S3, obtaining remaining hardware resources of the at least two processing hosts, sorting the at least two processing hosts according to the remaining hardware resources, so as to select the processing host having the most remaining hardware resources;
Step S4, allocating the processing host to the data acquisition unit, and generating a communication loop corresponding to the processing host and the data acquisition unit based on the network topology;
wherein the communication loop is the one in which the least number of the gateway devices pass between the processing host and the data acquisition unit.

Preferably, Step S4 further comprises:
Step S41, allocating the processing host with the most remaining hardware resources to the data acquisition unit;
Step S42, obtaining each of the plurality of gateway devices on the communication loop between the processing host and the data acquisition unit according to the network topology;
Step S43, obtaining a current load of the gateway device, and determining whether the gateway device satisfies a preset communication condition based on the current load;
if yes, turn to Step S45;
if no, turn to Step S44;
Step S44, selecting another gateway device, satisfying the preset condition and located adjacent to the gateway device, as the gateway device in the communication loop; and
Step S45, generating the communication loop according to the gateway device.

By adopting the above-mentioned technical solutions, the present invention has the beneficial effects that a data acquisition unit is provided to enable ultrasonic image data to effectively flow in a hospital or regional network, so that the problem of a poor flow of the ultrasonic image data in the prior art is avoided. A high-quality reconstruction of the ultrasonic image is carried out by a processing host, ensuring a volume of a scanning device is reduced without compromising the imaging quality, so that it is easy to move the scanning device.

### DESCRIPTION OF THE DRAWINGS

Embodiments of the present invention will be fully illustrated with reference to the accompanying drawings, however, which are given for the sake of clarification and description only, without limiting the scope of the invention.

FIG. 1 is a schematic diagram showing a principle of an embodiment of the present invention;

FIG. 2 is a schematic diagram showing a communication connection among terminal processing devices in an embodiment of the present invention;

FIG. 3 is a schematic diagram of each image processing module in an embodiment of the present invention;

FIG. 4 is a flow chart of a communication method in an embodiment of the present invention;

FIG. 5 is a schematic diagram showing a network connection in an embodiment of the present invention;

FIG. 6 is a schematic diagram showing sub-steps of Step S4 in an embodiment of the present invention; and

FIG. 7 is a schematic diagram showing a connection of a user terminal in an embodiment of the present invention.

### DETAILED DESCRIPTION

The technical solution set forth in the embodiments of the present invention will now be described clearly and fully hereinafter with reference to the accompanying drawings of the embodiments of the present invention. Obviously, such embodiments provided in the present invention are only part of the embodiments instead of all embodiments. It should be understood that all the other embodiments obtained from the embodiments set forth in the present invention by one skilled in the art without any creative work fall within the scope of the present invention.

Notably, the embodiments set forth in the present invention and features of the embodiments may be combined in any suitable manner.

The present invention will be described hereinafter with reference to the accompanying drawings and particular embodiments, but the invention is not limited thereto.

The invention comprises:
a distributed ultrasonic scanning system, as shown in FIG. 1, comprising:
at least one ultrasonic scanning device 1, comprising at least one scanning probe for performing an ultrasonic scanning operation on a patient to obtain echo data;
a data acquisition unit 2, connected to the at least one ultrasonic scanning device 1, configured to receive the echo data and to compress the echo data to generate compressed data; and
a processing host 3, connected to the data acquisition unit 2, configured to obtain the compressed data and to generate a first ultrasonic image according to the compressed data.

In particular, due to the fact that an ultrasonic scanning device in the prior art is large in size and is arranged in a fixed location, it cannot be moved as desired to be suited for scenarios where examining a patient in a ward is required. For this purpose, in this embodiment, echo data is obtained by providing the data acquisition unit 2, then image reconstruction is done by the processing host 3, so that a volume of the ultrasonic scanning device 1 is reduced, and thus mobility of the ultrasonic scanning device 1 is realized.

During the process of the detection, the ultrasonic scanning device 1 may be a hand-held ultrasonic scanning device, which has wired or wireless communication capabilities to connect to the data acquisition unit 2 through Ethernet, optical fiber, WLAN, cellular mobile network, radio frequency communication and other means, so as to send the echo data to the data acquisition unit 2. It should be noted that the echo data may be an original ultrasonic echo sequence obtained by the scanning probe, or it may be echo data after being subjected to data compression. The processing host 3 may be one or more general-purpose computers, application-specific integrated circuits (ASICs), DSPs, programmable logic devices (PLDs), complex programmable logic devices (CPLDs), field programmable gate arrays (FPGAs), general-purpose processors, controllers, microcontrollers (MCUs), microprocessors or other electronic components to achieve the above-mentioned communication and data processing functions. Upon receipt of the compressed data, the processing host 3 can decompress the compressed data to obtain the above-mentioned echo data, and to obtain a first ultrasonic image by reconstructing the echo data. The first ultrasonic image may be a single ultrasonic image, or a continuous ultrasound image with a preset time length. The information management unit 4 can be used as an embodiment of a software, which is a part of a hospital information system. The information management unit 4 obtains and stores the first ultrasonic image via the hospital or regional network, and distributes data to the medical terminals 5 according to a corresponding preset condition. The medical terminal 5 may be a computer installed in a specific healthcare institution, a department or at home, which is used for reading patient medical records, for issuing prescriptions, etc.

It should be noted that the gateway devices A1, the medical terminals 5 and other devices in FIG. 1 are only simple examples of information flow, and do not constitute a limitation on the quantity and actual connection relationship of various types of devices. For example, the medical terminals 5 can be connected to the information management unit through the plurality of gateway devices A1; the plurality of gateway devices A1 actually form a local area network, and the above-mentioned data acquisition unit 2, the processing host 3, the information management unit 4 and the medical terminals 5 access through any or particular node in the local area network to obtain corresponding data; the plurality of medical terminals 5 can access the information management unit 4 through the gateway devices A1 to obtain the first ultrasound image; the plurality of processing hosts 3 obtain compressed data in a specific order to separately or jointly generate the first ultrasound image; a plurality of data acquisition units 2 are provided as needed, which correspond to a plurality of ultrasonic scanning devices 1, and access the local area network through the same or the plurality of gateway devices 1; alternatively, the adjacent data acquisition unit 2, the adjacent information management unit 4 are directly connected to the processing post 3 without passing through the plurality of gateways devices A1, etc.

As an optional embodiment, after the processing host 3 completes the image reconstruction and forms the above-mentioned first ultrasound image, the first ultrasonic image can be sent to the information processing unit 4 through the gateway device A1, and then the information processing unit 4 stores the first ultrasound image, so that the first ultrasound image can be transmitted to the medical terminals 5 for review of doctors during the diagnosis and treatment process. Or, the processing host 3 transmits the first ultrasonic image to the a display screen B1 through the gateway device A1 to display the first ultrasonic image on the display screen B1.

During the process of detection, the display screen B1 may be a display screen in a mobile terminal, such as a mobile phone, a tablet or a notebook, which establishes a communication connection with the gateway device A1 to receive the first ultrasonic image sent from the processing host 3, or it may be other display screens with a network connection function, such as smart TVs. Alternatively, the display screen B1 may also be connected to the gateway device A1 through an image processing device with a network connection function (not shown in the figures), and the first ultrasonic image is displayed by converting an Ethernet signal into a video signal through the image processing device.

In a preferred embodiment, as shown in FIG. 1, the ultrasonic scanning system further comprises a terminal processing device 5;
wherein the terminal processing device 5 is connected to the data acquisition unit 2 or the ultrasonic scanning device 1 to obtain the echo data;
the terminal processing device 5 generates and displays a second ultrasonic image according to the echo data; and
an imaging quality of the second ultrasonic image is lower than that of the first ultrasonic image.

In particular, it is known that the ultrasonic scanning device in the prior art cannot be well adapted to the mobile scanning scene. In this embodiment, a terminal processing device 5 is added to the ultrasonic scanning system to realize image reconstruction of the ultrasonic echo data, and thus in a case where the processing host 3 is not connected, a quick and rough scan image can be obtained by reducing the imaging quality.

During the process of detection, the terminal processing device 5 may be a terminal device with basic graphics processing capabilities, such as a mobile phone, a tablet, a laptop or other devices with a GPU. Those terminal devices may be connected to the data acquisition unit 2 or the ultrasonic scanning device 1 in a wired or wireless manner, for example via a coaxial cable, a bus, an Ethernet, an optical fiber, WLAN or other radio frequency communication means, so as to receive the echo data. The reduction in imaging quality is mainly reflected in that at least one of the parameters, such as a resolution, a gray level, and a frame rate of the second ultrasonic image is inferior to those of the first ultrasonic image, thereby reducing the requirements for the graphics processing capability of the terminal processing device 5, so as to better adapt to the requirements of mobile scanning operations.

As an optional embodiment, the terminal processing device 5 may also receive the first ultrasonic image returned from the data acquisition unit 2 and generated by the processing host 3, so as to obtain a high-quality scan image nearby.

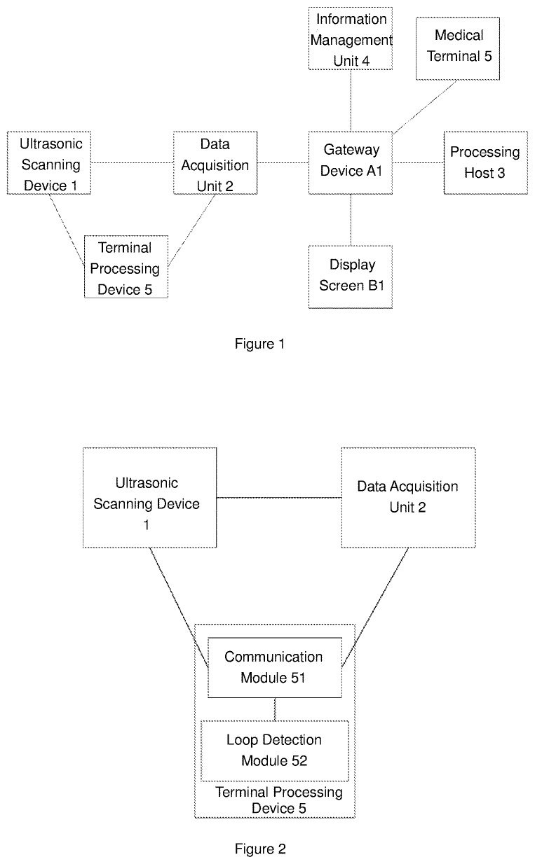

In a preferred embodiment, as shown in FIG. 2, the terminal processing device 5 comprises:
a communication module 51, connected to the ultrasonic scanning device 1 or the data acquisition unit 2, and configured to receive the echo data;
a loop detection module 52, connected to the communication module 51;
when the communication module 51 receives the echo data from the ultrasonic scanning device 1, the loop detection module 52 detects a communication connection status between the communication module 51 and the ultrasonic scanning device 1;
when the communication connection status reaches a preset condition, the loop detection module 52 controls the communication module 51 to establish a communication connection with the data acquisition unit 2, so as to continue to receive the echo data.

In particular, in the mobile scanning scenario, a wireless condition or transmission distance of a specific location cannot well meet the connection requirements between the terminal processing device 5 and the ultrasonic scanning device 1. Therefore, in this embodiment, the loop detection module 52 is added to the terminal processing device 5 for detecting the communication connection status. In addition, the connection is adjusted in real time according to the communication connection to obtain a stable image transmission effect. Or, the addition of the loop detection module 52 facilitates users far away from the ultrasonic scanning device 1 to carry out relay communication through the data acquisition unit 2, to remotely obtain a second ultrasonic image.

During the process of detection, the communication connection status comprises at least one of received signal strength, signal transmission delay, and packet loss rate, and the preset condition is a parameter threshold set for the communication connection status. When any corresponding parameter triggers the parameter threshold, the loop detection module 52 immediately controls the communication module 51 to establish the communication connection with the data acquisition unit 2.

As an optional embodiment, when the first ultrasonic image and the second ultrasonic image need to be generated at the same time, the ultrasonic scanning device 1 performs a handshake with the data acquisition unit 2 and the terminal processing device 5 respectively to determine a transmission identifier, and then performs broadcast communication in a specific frequency band to send the echo data to the data acquisition unit 2 and the terminal processing device 5 at the same time, thereby reducing the demand for the communication capability of the ultrasonic scanning device 1.

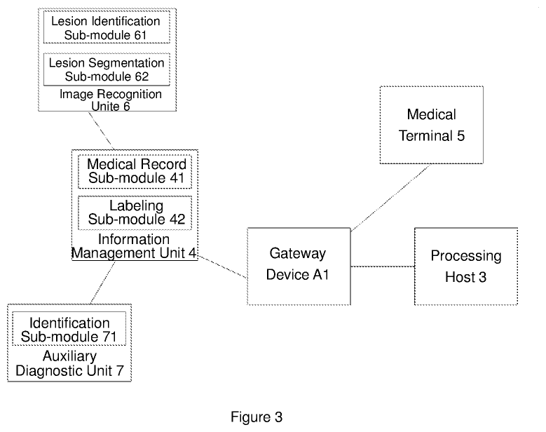

In a preferred embodiment, as shown in FIG. 3, the ultrasonic scanning system further comprises:
an information management unit 4, connected to the processing host 3, and configured to obtain the first ultrasonic image;
wherein the information management unit 4 comprises a medical record sub-module 41, the medical record sub-module 41 records the first ultrasonic image in a pre-collected electronic medical record corresponding to the patient.

In particular, in the prior art, results of ultrasonic diagnosis usually only comprise image screenshots and diagnostic conclusions issued by the imaging department. It cannot well meet the demands for medical data to be circulated in the whole hospital in a digital healthcare. In addition, doctors in other healthcare institutions or other departments or home users are not able to make further diagnosis according to the ultrasonic image. For this problem, in this embodiment, a medical record sub-module 41 is added to the information management unit 4 to record a high-quality first ultrasonic image in a patient's electronic medical record, so that the user can replay the first ultrasonic image according to actual needs, and thus can make a more accurate diagnosis.

In a preferred embodiment, as shown in FIG. 3, the ultrasonic scanning system further comprises an image recognition unit;
wherein the image recognition unit 6 is connected to the information management unit 4 for obtaining the first ultrasonic image from the information management unit 4;
the image recognition unit 6 comprises:
a lesion identification sub-module 61, configured to determine the presence of a lesion in the first ultrasonic image based on the first ultrasonic image;
a lesion segmentation sub-module 62, connected to the lesion identification sub-module 61;
when the lesion is present in the first ultrasonic image, the lesion segmentation sub-module segments an image of the lesion from the first ultrasonic image and stores the segmented lesion image in the information management unit 4.

In particular, the existing ultrasonic image diagnosis is mainly made by medial personnel, so it is quite time-consuming. In this embodiment, a quick detection of lesions in the first ultrasonic image is done by the provision of the image recognition unit 6, and the lesion image is separated from the first ultrasonic image to help doctors make a quick diagnosis, and it also provides a health alert for family users.

During the process of detection, the lesion identification sub-module 61 has a lesion identification model. The lesion identification model selects a plurality of manually annotated lesion images as a training set, and can effectively identify the size and position of a target lesion, and then generates lesion information. The lesion segmentation module 62 may be a semantic segmentation model, which further separates the lesion image from the first ultrasonic image through the obtained lesion information.

In a preferred embodiment, as shown in FIG. 3, the ultrasonic scanning system further comprises an auxiliary diagnostic unit 7;
wherein the auxiliary diagnostic unit 7 is connected to the information management unit 4 for obtaining the lesion image from the information management unit 4;
the auxiliary diagnostic unit 7 comprises an identification sub-module 71 for identifying the lesion in the lesion image to generate classification information;
the medical record sub-module 41 in the information management unit 4 receives and stores the classification information, and the classification information is used to assist the doctor in diagnosing the patient and to provide an individual health alert for family users.

In particular, the diagnosis of lesion morphology in the prior art mainly relies on medical personnel and takes up a lot of human resources, in this embodiment, an auxiliary diagnostic unit 7 is added to realize a rapid judgment of the lesion morphology in the lesion image, thereby helping the doctor determine the disease progress according to the lesion morphology, and helping providing an individual health alert for family users.

During the process of detection, the identification sub-module 71 is provided with a morphological artificial intelligence model, which can determine the current shape of the lesion according to the image of the lesion, and compares the current shape with a plurality of preset lesion morphology corresponding to the disease progress to generate a confidence level of each morphology as the classification information, thereby help users judge the morphology of the current lesion.

In a preferred embodiment, the information management unit 4 is connected to a plurality of medical terminals 5;
the information management unit 4 selects a corresponding one of the plurality of medical terminals 5 according to the electronic medical record and a pre-entered diagnosis process, so as to distribute at least one data of the electronic medical record, the first ultrasonic image and the lesion image.

In particular, in order to solve the problem of a relatively unsmooth flow of ultrasonic images in the prior art, in this embodiment, the information management unit distributes data to the corresponding medical terminal 5 to realize a rapid flow of overall medical record data and ultrasonic images. As a result, each user in the patient's diagnostic process can obtain the corresponding data in time.

In a preferred embodiment, each of the plurality of medical terminals 5 has a corresponding healthcare institution identifier and a hospital department identifier;
wherein the information management unit 4 comprises a labeling sub-module 42, the labeling sub-module 42 obtains the healthcare institution identifier and the hospital department identifier of one of the plurality of medical terminals 5, and selects, based on the healthcare institution identifier and the hospital department identifier, a corresponding process flow to process the first ultrasonic image; and
the information management unit distributes the processed first ultrasonic image to the medical terminals 5.

In particular, in order to achieve high circulation and readability of the medical record data, in this embodiment, the labeling sub-module 42 is added to the information management unit 4 to realize labeling processing of the first ultrasonic image, thereby facilitating the user to read out relevant information in a more clear way after he/she obtains an ultrasonic image. In addition, data labeling requirements corresponding to different healthcare institutions and departments are preset in the labeling sub-module 42, and more effective labeling information can be generated by acquiring the healthcare institution identifier and department identifier of the medical terminal and by selecting the corresponding processing flow to process.

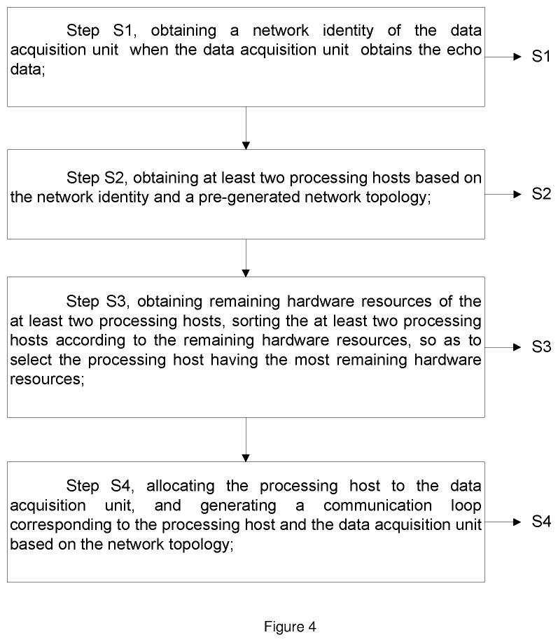

A communication method applicable for the ultrasonic scanning system as described above, wherein the data acquisition unit in the ultrasonic scanning system is connected to the processing host through a plurality of gateway devices, as shown in FIG. 4, the communication method comprises:
Step S1, obtaining a network identity of the data acquisition unit 2 when the data acquisition unit 2 obtains the echo data;
Step S2, obtaining at least two processing hosts 3 based on the network identity and a pre-generated network topology;
Step S3, obtaining remaining hardware resources of the at least two processing hosts 3, sorting the at least two processing hosts 3 according to the remaining hardware resources, so as to select the processing host 3 having the most remaining hardware resources;
Step S4, allocating the processing host 3 to the data acquisition unit 2, and generating a communication loop corresponding to the processing host 3 and the data acquisition unit 2 based on the network topology;
wherein the communication loop is the one in which the least number of the gateway devices pass between the processing host and the data acquisition unit.

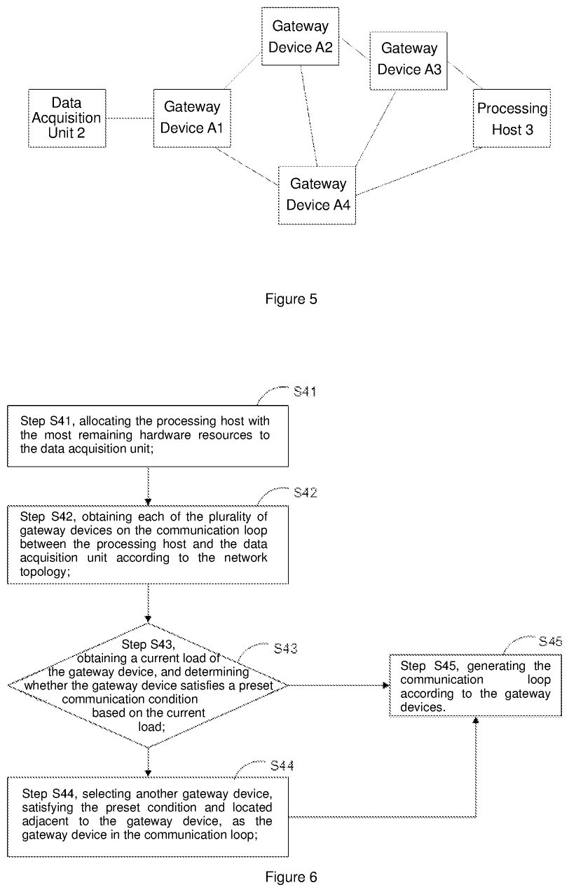

In particular, as shown in FIG. 5, in order to solve the problem that the network topology of the local area network is relatively complex and the transmission delay is large, in this embodiment, a processing method is provided. In this method, gateway devices A1, A2, A3, A4 in the network topology are detected to determine the shortest transmission path between the data acquisition unit 2 and the processing host 3, and then the shortest transmission path is adopted as an actual communication loop, thereby reducing the delay caused by the transmission path and reducing an overall load of the local area network.

In the meantime, the position information of the processing host 3 and the data acquisition unit 2 is also recorded in the network identifier. Processing hose 3 and the data acquisition unit 2, which are relatively close, are selected based on the relative position information therebetween, so that delay caused by the transmission can be reduced.

Furthermore, for the situation where many-to-many communication between the data acquisition unit 2 and the processing host 3 exists, this communication method also comprises a load balancing process. The load balancing process involves acquiring the remaining hardware resources of the processing host for allocating the processing host 3 to the data acquisition unit 2 according to the status of the remaining hardware resources, thereby the problem that a sudden scanning demand cannot be met due to an excessive load of parts of the processing hosts 3 can be avoided.

In a preferred embodiment, as shown in FIG. 6, Step S4 further comprises:
Step S41, allocating the processing host 3 with the most remaining hardware resources to the data acquisition unit 2;
Step S42, obtaining each of the plurality of gateway devices on the communication loop between the processing host 3 and the data acquisition unit 2 according to the network topology;
Step S43, obtaining a current load of the gateway device A1, A2, A3, A4, and determining whether the gateway device A1, A2, A3, A4 satisfies a preset communication condition based on the current load;
if yes, turn to Step S45;
if no, turn to Step S44;
Step S44, selecting another gateway device A1, A2, A3, A4, satisfying the preset condition and located adjacent to the gateway device A1, A2, A3, A4, as the gateway device A1, A2, A3, A4 in the communication loop; and
Step S45, generating the communication loop according to the gateway devices A1, A2, A3, A4.

In particular, since a large amount of data is exchanged over the local area network, in this embodiment, a step of a load condition detection for the gateway devices A1, A2, A3, and A4 is added. The load condition detection makes it possible to bypass the gateway devices A1, A2, A3, and A4 with excessive loads, so that interference with other communication services on the gateway devices A1, A2, A3, and A4 from compressed data with a relatively large amount of transmission data involved is avoided.

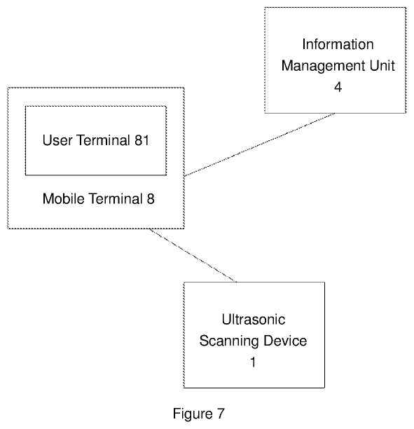

In a preferred embodiment, as shown in FIG. 7, the above-mentioned distributed ultrasonic scanning system further comprises:
a user terminal 81 arranged in a mobile terminal 8 of a patient;
wherein the user terminal 81 is connected to the information management unit 4 and acquires at least one data of the electronic medical record, the first ultrasonic image and the lesion image from the information management unit 4.

In particular, in view of the problem that the ultrasonic scanning device in the prior art cannot well satisfy the need for the patient to perform remote diagnosis at home or in the community, in this embodiment, the user terminal 81 is arranged in the patient's mobile terminal 8 to realize a remote medical treatment for the patient.

During the process of detection, the user terminal 81 is arranged in the mobile terminal 8 as a software embodiment. The mobile terminal 8 may be a general-purpose computing device with a communication function, such as a patient's mobile phone and a tablet. It is configured to connect to an inter-hospital system via the network, and obtain an electronic medical record, a first ultrasonic image and a lesion image of the patient. The electronic medical record contains some relevant examination processes and diagnosis conclusions during the patient's stay in the hospital for diagnosis, providing effective evidence for home-based medical care.

In a preferred embodiment, the user terminal 81 is also connected to the ultrasonic scanning device 1;
the user terminal 81 obtains echo data from the ultrasonic scanning device 1; and
the user terminal 81 generates a second ultrasonic image based on the echo data.

In particular, since the ultrasonic scanning device 1 in the prior art is not sufficient to meet the needs for patients to perform an ultrasonic scanning operation at home or in a community, in this embodiment, a rapid reconstruction of the echo data of the ultrasonic scanning device is achieved through the user terminal 81, thus, a basic simple scanning need is met. When the patients need a first scanning image with higher quality in the community, they can remotely obtain the first ultrasonic image generated from the processing host 3 through the information management unit 4.

The present invention has the beneficial effects that a data acquisition unit is provided to enable ultrasonic image data to effectively flow in the network, so that the problem of a poor flow of the image is avoided. Here, a high-quality reconstruction of the ultrasonic image is carried out by a processing host, ensuring a volume of a scanning device is reduced without compromising the imaging quality. In addition, an image reconstruction of data, obtained by a plurality of scanning probes, is carried out by a processing host, so that a centralized processing of images is achieved, and hardware costs are reduced.

The above descriptions are only the preferred embodiments of the invention, not thus limiting the embodiments and scope of the invention. Those skilled in the art should be able to realize that the schemes obtained from the content of specification and drawings of the invention are within the scope of the invention.

It is claimed:

1. A distributed ultrasonic scanning system, comprising:
at least one ultrasonic scanning device, comprising at least one scanning probe for performing an ultrasonic scanning operation on a patient to obtain echo data;
a data acquisition unit, connected to the at least one ultrasonic scanning device, configured to receive the echo data and to compress the echo data to generate compressed data; and
a processing host, connected to the data acquisition unit, configured to obtain the compressed data and to generate a first ultrasonic image according to the compressed data.

2. The ultrasonic scanning system of claim 1, wherein the ultrasonic scanning system further comprises a terminal processing device;
wherein the terminal processing device is connected to the data acquisition unit or the ultrasonic scanning device to obtain the echo data;
the terminal processing device generates and displays a second ultrasonic image according to the echo data; and
an imaging quality of the second ultrasonic image is lower than that of the first ultrasonic image.

3. The ultrasonic scanning system of claim 2, wherein the terminal processing device comprises:
a communication module, connected to the ultrasonic scanning device or the data acquisition unit, and configured to receive the echo data;
a loop detection module, connected to the communication module;
when the communication module receives the echo data from the ultrasonic scanning device, the loop detection module detects a communication connection status between the communication module and the ultrasonic scanning device;
when the communication connection status reaches a preset condition, the loop detection module controls the communication module to establish a communication connection with the data acquisition unit, so as to continue to receive the echo data.

4. The ultrasonic scanning system of claim 1, wherein the ultrasonic scanning system further comprises:
an information management unit, connected to the processing host, and configured to obtain the first ultrasonic image;
wherein the information management unit comprises a medical record sub-module, the medical record sub-module records the first ultrasonic image in a pre-collected electronic medical record corresponding to the patient.

5. The ultrasonic scanning system of claim 4, wherein the ultrasonic scanning system further comprises an image recognition unit;
wherein the image recognition unit is connected to the information management unit for obtaining the first ultrasonic image from the information management unit;
the image recognition unit comprises:
a lesion identification sub-module, configured to determine the presence of a lesion in the first ultrasonic image based on the first ultrasonic image;
a lesion segmentation sub-module, connected to the lesion identification sub-module;
when the lesion is present in the first ultrasonic image, the lesion segmentation sub-module segments an image of the lesion from the first ultrasonic image and stores the segmented lesion image in the information management unit.

6. The ultrasonic scanning system of claim 5, wherein the ultrasonic scanning system further comprises an auxiliary diagnostic unit;
wherein the auxiliary diagnostic unit is connected to the information management unit for obtaining the lesion image from the information management unit;
the auxiliary diagnostic unit comprises an identification sub-module for identifying the lesion in the lesion image to generate classification information;
the medical record sub-module in the information management unit receives and stores the classification information, and the classification information is used to assist the doctor in diagnosing the patient.

7. The ultrasonic scanning system of claim 5, wherein the information management unit is connected to a plurality of medical terminals;
the information management unit selects a corresponding one of the plurality of medical terminals according to the electronic medical record and a pre-entered diagnosis process, so as to distribute at least one data of the electronic medical record, the first ultrasonic image and the lesion image.

8. The ultrasonic scanning system of claim 7, wherein each of the plurality of medical terminals has a corresponding healthcare institution identifier and a hospital department identifier;
wherein the information management unit comprises a labeling sub-module, the labeling sub-module obtains the healthcare institution identifier and the hospital department identifier of one of the plurality of medical terminals, and selects, based on the healthcare institution identifier and the hospital department identifier, a corresponding process flow to process the first ultrasonic image; and
the information management unit distributes the processed first ultrasonic image to the medical terminals.

9. The ultrasonic scanning system of claim 5, wherein the ultrasonic scanning system further comprises:
a user terminal arranged in a mobile terminal of a patient;
wherein the user terminal is connected to the information management unit and acquires at least one data of the electronic medical record, the first ultrasonic image and the lesion image from the information management unit.

10. The ultrasonic scanning system of claim 9, wherein the user terminal is also connected to the ultrasonic scanning device;
the user terminal obtains echo data from the ultrasonic scanning device; and
the user terminal generates a second ultrasonic image based on the echo data.

11. A communication method applicable for the ultrasonic scanning system of claim 1, wherein the data acquisition unit in the ultrasonic scanning system is connected to the processing host through a plurality of gateway devices, the communication method comprises:
Step S1, obtaining a network identity of the data acquisition unit when the data acquisition unit obtains the echo data;
Step S2, obtaining at least two processing hosts based on the network identity and a pre-generated network topology;
Step S3, obtaining remaining hardware resources of the at least two processing hosts, sorting the at least two processing hosts according to the remaining hardware resources, so as to select the processing host having the most remaining hardware resources;
Step S4, allocating the processing host to the data acquisition unit, and generating a communication loop corresponding to the processing host and the data acquisition unit based on the network topology;
wherein the communication loop is the one in which the least number of the gateway devices pass between the processing host and the data acquisition unit.

12. The communication method of claim 11, wherein Step S4 further comprises:
Step S41, allocating the processing host with the most remaining hardware resources to the data acquisition unit;
Step S42, obtaining each of the plurality of gateway devices on the communication loop between the processing host and the data acquisition unit according to the network topology;
Step S43, obtaining a current load of the gateway device, and determining whether the gateway device satisfies a preset communication condition based on the current load;
if yes, turn to Step S45;
if no, turn to Step S44;
Step S44, selecting another gateway device, satisfying the preset condition and located adjacent to the gateway device, as the gateway device in the communication loop; and Step S45, generating the communication loop according to the gateway device.
